(19) United States
(12) Patent Application Publication
(10) Pub. No.: US 2006/0008433 A1
(43) Pub. Date: Jan. 12, 2006

(54) Cosmetic emulsions containing liquid-crystal dyes

(75) Inventor: Gaetan Chevalier, Boigny Sur Bionne (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/172,977

(22) Filed: Jul. 5, 2005

(30) Foreign Application Priority Data
Jul. 5, 2004 (FR) 04 51435

(51) Int. Cl. A61K 8/89; A61K 8/49

(52) U.S. Cl. 424/61; 424/63; 424/70.12

(57) ABSTRACT

A emulsion-type cosmetic composition for caring for and/or making up the skin, mucous membranes and/or the integuments, includes at least one liquid continuous phase; and at least one liquid dispersed phase. The composition includes at least one volatile solvent; the dispersed liquid phase includes at least one dye with a liquid-crystal colored phase; and the at least one dye is present in a concentration such that, after the composition is dried on a support, a dried concentration of the dye is greater than or equal to about 50% by weight relative to a total weight of the dried composition. A cosmetic process and a makeup kit are also provided.

### CROSS-REFERENCE TO RELATED APPLICATION

This application claims the benefit of French Application No. 04 51435, filed on Jul. 5, 2004, the entire disclosure of which is incorporated by reference herein.

### BACKGROUND

The present invention relates to cosmetic compositions containing dyes of liquid-crystal type, and especially dyes having a chiral nematic phase. Such cosmetic compositions may be used for making up skin of the human face or body, lips and integuments, such as nails, eyelashes, eyebrows or hair.

Known makeup compositions contain dyestuffs to give a final deposit (i.e., after application and drying of the composition on the surface to be made up) the desired color.

Dyestuffs usually used are organic pigments such as lakes or mineral pigments, especially nacres.

To obtain novel color effects, it has been proposed to use in makeup compositions more elaborate dyestuffs, for instance interference pigments and certain coloring agents with liquid-crystal phases.

U.S. Pat. No. 5,851,277 and EP 815 826 propose makeup compositions comprising interference pigments such as polyorganosiloxanes comprising liquid-crystal groups. Interference pigments are also described in U.S. Pat. Nos. 4,362,315 and 5,851,604. These interference pigments produce a color in a shade range between at least two specific colors that varies as a function of the incidence of light and the angle of observation. Such pigments are sold, for example, under the name HELICONE by the company Wacker, also known under the CTFA name Polysilicone-12.

U.S. Pat. No. 6,346,237 proposes cosmetic compositions comprising coloring agents of liquid-crystal type, such as silicones or cellulose ethers to which mesomorphic groups such as cholesterol-based units or units of biphenyl type are grafted, in combination with conventional coloring agents.

### SUMMARY

In various exemplary embodiments, emulsion-type cosmetic compositions for caring for and/or making up the skin, mucous membranes and/or the integuments, include: at least one liquid continuous phase; and at least one liquid dispersed phase. In various exemplary embodiments, the composition comprises at least one volatile solvent; the dispersed liquid phase comprises at least one dye with a liquid-crystal colored phase; and the at least one dye is present in a concentration such that, after the composition is dried on a support, a dried concentration of the dye is greater than or equal to about 50% by weight relative to a total weight of the dried composition.

In various exemplary embodiments, cosmetic processes for making up the skin, mucous membranes and/or the integuments, include: applying a first coat of a first cosmetic composition to a surface to be made up; and applying a second coat of a second cosmetic composition over the first coat. In various exemplary embodiments, the first cosmetic composition comprises at least one dyestuff in a physiologically acceptable medium, the first cosmetic composition being free of dyes with liquid-crystal colored phases; and the second cosmetic composition is an emulsion-type composition comprising at least one liquid continuous phase and at least one liquid dispersed phase, the second composition comprising at least one volatile solvent and the liquid dispersed phase comprising an effective amount of at least one dye with a liquid-crystal colored phase.

In various exemplary embodiments, makeup kits, include: a first cosmetic composition comprising at least one dyestuff in a cosmetically acceptable medium, the first composition being free of dyes with liquid-crystal colored phases; and a second emulsion-type cosmetic composition comprising at least one liquid continuous phase and at least one liquid dispersed phase, the second cosmetic composition comprising at least one volatile solvent and the liquid dispersed phase comprising an effective amount of at least one dye with a liquid-crystal colored phase. In various exemplary embodiments, the first and second compositions are packaged in separate containers.

### DETAILED DESCRIPTION OF EMBODIMENTS

The present inventors have discovered that it is possible to obtain makeup with a novel optical effect, using dyes with liquid-crystal colored phases, including dyes having a chiral nematic phase.

The first liquid crystals with a chiral nematic phase were observed in cholesterol-based compounds. Initially, these types of crystals were also termed cholesterol-phase liquid crystals. However, it has since been discovered that compounds in which the cholesterol function is absent from the chemical structure can have a cholesterol phase or, more particularly, a chiral nematic phase.

The term “chiral nematic phase” refers to a mode of organization of liquid-crystal molecules, in solid form or as a highly concentrated solution, in a three-dimensional space.

The main characteristics of the nematic phase are the following: molecules have a tendency to align in parallel with each other, leading to an anisotropic orientation; the nematic phase is a medium with only one axis of orientation; and there is no particular order in the position of the centers of gravity of molecules, which advantageously gives the nematic phase fluidity. If constituent molecules comprise at least one asymmetric carbon, a chiral nematic phase will result, which can provide an optical effect.

Chiral nematic phases are characterized by selective reflection of light. This reflection generates a multicolored and/or iridescent texture. This iridescence is readily observable by the naked eye with white light. When observed using a polarized-light microscope, these phases show birefringence. Dyes with a liquid-crystal colored phase and with a chiral nematic phase are particularly effective at displaying this type of optical phenomenon.

This effect is particularly advantageous for obtaining dynamic makeup. Moreover, the effect may be advantageous for attenuating and/or concealing local imperfections of the skin or the lips, for instance color stains, redness, pores and fine lines.

However, these optical phenomena can only be manifested when molecules are present in solid and/or highly concentrated form, e.g., in a composition with a liquid crystal concentration of greater than or equal to 60% by weight relative to a total weight of a final deposit.

However, for obvious reasons, such high concentrations pose problems in terms of formulation in cosmetic compositions.

In addition to this limitation in terms of concentration, dyes with a liquid-crystal colored phase generally have a natural consistency like extremely tacky very viscous liquids in dry form, irrespective of their chemical nature.

In various exemplary embodiments, the present invention provides solutions to problems raised by the formulation of cosmetic compositions including dyes with a liquid-crystal colored phase in amounts sufficient to give a support the expected optical effect after application of these compositions to a surface of the support.

In various exemplary embodiments, the present invention relates to cosmetic compositions for caring for and/or making up the skin, mucous membranes and/or the integuments, especially the nails, of emulsion type, comprising at least one liquid continuous phase and at least one liquid dispersed phase, characterized in that the emulsion contains at least one volatile solvent and the dispersed liquid phase contains an effective amount of at least one dye with a liquid-crystal colored phase, the concentration of which is adjusted to obtain, after application and drying of said composition on a support, a concentration of dye with a liquid-crystal colored phase of greater than or equal to 50% by weight relative to the total weight of the final deposit on the support.

In various exemplary embodiments, the present invention further includes cosmetic processes for making up the skin, mucous membranes or the integuments, especially the nails, comprising the application of a composition as defined above to the skin, mucous membranes or the integuments.

In various exemplary embodiments, the present invention includes cosmetic processes for making up the skin, mucous membranes and/or the integuments, comprising the application to the surface to be made up of a first coat (also known as a base coat) of a first cosmetic composition comprising, in a physiologically acceptable medium, at least one dyestuff, followed by the application, onto at least a part of said first coat, of a second coat (also known as a surface coat) of a second cosmetic composition in accordance with the invention, the first composition being free of dye with a liquid-crystal colored phase.

In various exemplary embodiments, makeup kits include a first cosmetic composition (also known as a base composition) comprising, in a cosmetically acceptable medium, a first dyestuff, and a second cosmetic composition (also known as a surface composition) in accordance with the invention. The first composition may be free of dye with a liquid-crystal colored phase. The first and second compositions may be packaged in separate containers.

In various exemplary embodiments, made-up supports include a makeup obtained using a composition in accordance with the invention applied to the support. The support may be chosen from false nails, false eyelashes, wigs, hairpieces, and dots or patches that adhere to the skin or the lips.

Dyes with a Liquid Crystal Colored Phase

As used herein, a dye with a liquid crystal colored phase includes any substance capable of leading to a colored effect with a liquid-crystal phase. This effect may be characterized by birefringence and a multicolored and/or iridescent texture.

Birefringence may be characterized by observation, using a polarized-light microscope, of a sample of a composition containing such a dye. This birefringence may be monitored, for example, using a Leica DMLB microscope.

Multicolored and/or iridescent texture may be seen using the naked eye.

Such effects are manifest in dyes with a liquid crystal colored phase containing a chiral nematic phase as defined above.

Such optical effects are generally observed only at and above certain concentrations of dyes with a liquid-crystal colored phase. Such a concentration is referred to herein as an “effective amount.” An “effective amount” of a dye with a liquid-crystal colored phase and/or with a chiral nematic phase, is an amount that is sufficient to give such optical effects to a final deposit, obtained after application and drying of a composition containing the dye.

For obvious reasons, effective amounts will vary significantly depending on the nature of a dye and/or the weight proportion of a volatile phase in a composition.

Concentrations may be adjusted to give a final deposit, obtained after application and drying of a composition on a surface, a concentration of dyes with a liquid-crystal colored phase of, for example, greater than or equal to 50% by weight, greater than or equal to 55% by weight, greater than or equal to 60% by weight, from 60% to 80% by weight and from 64% to 75% by weight relative to a weight of the final deposit.

A final deposit corresponds to a nonvolatile fraction of a composition, i.e., all of the constituents present in the composition that are not volatile. The term “volatile compound” refers a compound which, taken in isolation, has a vapor pressure, at room temperature (25° C.) and atmospheric pressure, ranging from 10⁻³ to 300 mmHg (0.133 Pa to 40 000 Pa).

In other words, a nonvolatile fraction of a composition corresponds to a mixture of constituents remaining on skin after total drying of the composition applied to the skin.

In various exemplary embodiments, dyes with a liquid-crystal colored phase are heat-sensitive. For example, such dyes are liable to change color or to acquire a color in response to a temperature change. This capacity may be particularly advantageous in terms of makeup since it is capable of giving a certain dynamic aspect to makeup.

In various exemplary embodiments, dyes are liposoluble or organosoluble. In such embodiments, dyes may be formulated in a dispersed phase of oil type.

In various exemplary embodiments, water-soluble dyes with a liquid-crystal colored phase may be employed. Such water-soluble dyes may be formulated in an aqueous or aqueous-alcoholic dispersed phase, for example of polyol type.

Synthetic dyes with a liquid-crystal colored phase may be preferable to natural cholesterol-based dyes with a liquid-crystal colored phase, which have the drawback of being very viscous and thus more difficult to formulate, and for which it is more difficult to ensure total harmlessness.

In various exemplary embodiments, dyes are not silicone-based. Such dyes may have a chiral nematic liquid-crystal colored phase. Such dyes may be used in the form of a mixture combining several dyes.

Exemplary dyes with a liquid-crystal colored phase that may be suitable for use in the compositions described herein include, but are not limited to, dyes sold by the company Hallcrest under the reference BN.

Exemplary dyes include BN 533, BN 600, BN 825, BN 826, BN 823 and BN 1001.

BN 533 is composed of the following ingredients: methylbutylphenyl hexyloxybenzoate, methylbutylphenyl decyloxybenzoate, methylbutylphenyl octyloxy-benzoate, methylbutylphenyl dodecyloxybenzoate, methylbutylphenyl heptylbiphenyl-carboxylate, methylbutylphenyl pentylbenzoate and methylbutylphenyl propylbenzoate.

BN 600 is composed of the following ingredients: pentylphenyl methoxybenzoate, methylbutylphenyl hexyloxybenzoate, methylbutylphenyl heptylbiphenyl-carboxylate and methylbutylphenyl octyloxybenzoate.

BN 825 is composed of the following ingredients: pentylphenyl methoxybenzoate, methylbutylphenyl octyloxybenzoate, methylbutylphenyl hexyloxy-benzoate, methylbutylphenyl heptylbiphenylcarboxylate and methylbutylphenyl propyl-benzoate.

BN 826 is composed of the following ingredients: pentylphenyl methoxybenzoate, methylbutylphenyl octyloxybenzoate, methylbutylphenyl hexyloxy-benzoate, methylbutylphenyl heptylbiphenylcarboxylate and methylbutylphenyl propyl-benzoate.

BN 823 is composed of the following ingredients: pentylphenyl methoxy-benzoate, methylbutylphenyl hexyloxybenzoate, methylbutylphenyl octyloxybenzoate and methylbutylphenyl heptylbiphenylcarboxylate.

BN 1001 is composed of the following ingredients: methylbutylphenyl octyloxybenzoate, methylbutylphenyl decyloxybenzoate, methylbutylphenyl hexyloxy-benzoate, pentylphenyl methoxybenzoate and pentylphenyl octyloxybenzoate.

Exemplary dyes with a liquid-crystal colored phase may, where appropriate, be combined with other more conventional dyestuffs, such as identified below.

When these additional dyestuffs are formulated in a liquid phase separate from that containing the dyes with a liquid-crystal colored phase, i.e., a continuous phase, the dyestuffs may be present in very variable proportions.

When dyes and additional dyestuffs are formulated in the same phase, i.e., a continuous phase, it is preferable for the dyes with a liquid-crystal colored phase to be present in a greater proportion.

Concentrations of additional dyestuffs may, for example, not exceed 10% of a total weight of the dye mixture, i.e., dye(s) with a liquid-crystal colored phase and additional dyestuff(s).

Dyes with a liquid-crystal colored phase may be present, for example, in a proportion of more than 60% by weight relative to a total weight of the dye mixture.

Emulsion

As used herein, the term “emulsion” encompasses any medium comprising at least two liquid phases that are immiscible at room temperature and atmospheric pressure. This may include a direct emulsion (water-in-oil), an inverse emulsion (oil-in-water) or a multiple emulsion.

Emulsions composed of several phases dispersed in a single continuous phase are also encompassed.

The present inventors have discovered, unexpectedly, that formulation of dyes with a liquid-crystal colored phase in a dispersed liquid phase of an emulsion, generally containing at least one volatile compound, advantageously makes it possible to formulate an amount of these dyes that is sufficient to obtain a desired optical effect for a final deposit on a makeup support, while at the same time preserving satisfactory cosmetic qualities of the deposit.

Exemplary compositions contain at least one volatile compound in its dispersed phase containing a dye with a liquid-crystal colored phase. Such volatile compounds generally contribute to dissolution of dyes.

Volatile compounds may be present in a dispersed phase and in a continuous phase. For obvious reasons, such volatile compounds will be different. A dispersed phase, or even a continuous phase, may consist solely of a volatile solvent or mixture of volatile solvents.

As used herein, “volatile compound” refers to any compound capable of evaporating on contact with skin in less than one hour, at room temperature and atmospheric pressure.

A dispersed phase containing dyes with a liquid-crystal colored phase may be an oily, aqueous or aqueous-alcoholic phase depending on the chemical nature of the selected dye. In such a case, a volatile solvent will also be selected so as to allow dissolution of a dye in a dispersed phase.

For example, a dispersed phase may be of oily nature and a continuous phase of aqueous or aqueous-alcoholic nature. However, it may also be envisioned for a continuous phase also to be oily, while remaining immiscible with an oily phase forming a dispersed phase. In such a case, an emulsion is an oil in oil emulsion. Such an oil in oil emulsion may be a mixture of a hydrocarbon-based oil with a silicone oil.

Exemplary compositions may include emulsions in which a dye is formulated in an oily phase consisting totally or partially, for example, of isododecane, which is dispersed in an aqueous-alcoholic phase, for example based on a polyvinyl alcohol. In such a case, volatile compounds may include water and isododecane.

Consistencies of emulsions may be very variable, i.e., fluid, viscous, of gel type, or even solid like sticks.

Aqueous Phase

An aqueous phase may form a continuous phase or a dispersed phase in exemplary compositions.

Aqueous phases may consist essentially of water.

Aqueous phases may also comprise a mixture of water and of a water-miscible organic solvent (miscible in water to greater than 50% by weight at 25° C.), such as, for example, lower monoalcohols containing from 1 to 5 carbon atoms, such as ethanol, isopropanol, glycols containing from 2 to 8 carbon atoms, such as propylene glycol, ethylene glycol, 1,3-butylene glycol, dipropylene glycol, C₃-C₄ ketones and C₂-C₄ aldehydes.

An aqueous phase (water and optionally the water-miscible organic solvent) may be present in a content ranging, for example, from 1% to 95% by weight, from 5% to 80% by weight, and from 15% to 60% by weight, relative to a total weight of a composition.

When an aqueous phase constitutes a dispersed phase of an exemplary composition, it may comprise as a volatile solvent a short alcohol, e.g., an alcohol of less than C₁₀, and/or a volatile glycol such as propylene glycol.

Such a medium may also comprise a volatile oil as defined below.

Fatty Phase

A fatty phase may comprise at least one fatty substance that is liquid at room temperature (25° C.), where appropriate combined with a fatty substance that is solid at room temperature, such as waxes, pasty fatty substances and gums, and mixtures thereof. A fatty phase may also contain lipophilic organic solvents.

Exemplary compositions may advantageously contain, for example, a dispersed fatty phase.

An exemplary fatty phase may especially comprise, as a liquid fatty substance, at least one volatile or nonvolatile oil or a mixture thereof.

As used herein, the term “volatile oil” encompasses any oil capable of evaporating on contact with the skin in less than one hour, at room temperature and atmospheric pressure. Exemplary volatile oils are volatile cosmetic oils, which are liquid at room temperature, having a nonzero vapor pressure, at room temperature and atmospheric pressure, ranging, for example, from 0.01 to 300 mmHg (1.33 Pa to 40 000 Pa) and/or greater than 0.3 mmHg (30 Pa).

As used herein, the term “nonvolatile oil” refers to an oil that remains on the skin at room temperature and atmospheric pressure for at least several hours and that especially has a vapor pressure of less than 0.01 mmHg (1.33 Pa).

Volatile or nonvolatile oils may include hydrocarbon-based oils, silicone oils or mixtures thereof. The term “hydrocarbon-based oil” refers to an oil mainly containing hydrogen and carbon atoms and possibly oxygen, nitrogen, sulfur and phosphorus atoms.

Exemplary volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names ISOPAR and PERMETHYL, branched C₈-C₁₆ esters such as isohexyl neopentanoate, and mixtures thereof. Other volatile hydrocarbon-based oils, for instance petroleum distillates, especially those sold under the name SHELL SOLT by the company Shell, may also be used.

Volatile oils that may also be used include, but are not limited to, volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. Exemplary volatile silicone oils include, but are not limited to, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Nonvolatile oils may be chosen, for example, from nonvolatile hydrocarbon-based, fluoro and/or silicone oils.

Exemplary nonvolatile hydrocarbon-based oils include, but are not limited to:
hydrocarbon-based oils of animal origin;
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, blackcurrant oil, evening primrose oil, millet oil, barley oil, quinoa oil, rye oil, safflower oil, candlenut oil, passionflower oil or musk rose oil; shea butter; or caprylic/capric acid triglycerides, for instance those sold by the company Stéarineries Dubois or those sold under the names MIGLYOL 810, 812 and 818® by the company Dynamit Nobel;
synthetic ethers containing from 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol heptanoates, octanoates, decanoates or ricinoleates, for instance propylene glycol dioctanoate; hydroxylated esters, for instance isostearyl lactate or diisostearyl malate; polyol esters and pentaerythritol esters;
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol; and
higher fatty acids such as oleic acid, linoleic acid or linolenic acid, and mixtures thereof.

Exemplary nonvolatile silicone oils include, but are not limited to, nonvolatile polydimethylsiloxanes (PDMS), polydimethylsiloxanes comprising alkyl or alkoxy groups, which are pendant and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

Nonvolatile oils may be present in exemplary compositions a contents ranging, for example, from 0.01% to 90% by weight, from 0.1% to 85% by weight and from 1% to 70% by weight relative to a total weight of a composition.

In various exemplary embodiments, a liquid fatty phase comprising a volatile oil(s) may be present, for example, in a proportion of from 0.01% to 90% by weight and/or from 0.1% to 85% by weight relative to a weight of the composition.

As regards fatty substances that are solid at room temperature and atmospheric pressure, such substances may be chosen, for example, from waxes, pasty fatty substances and gums, and mixtures thereof. Fatty substances may be present, for example, in a proportion of from 0.01% to 50%, from 0.1% to 40% and/or from 0.2% to 30% by weight relative to a total weight of the fatty phase.

Thus, exemplary compositions may comprise at least one fatty substance that is pasty at room temperature.

As used herein, the term “pasty fatty substance” refers to fatty substances with a melting point ranging from 20 to 55° C. and preferably 25 to 45° C., and/or a viscosity at 40° C. ranging from 0.1 to 40 Pa·s (1 to 400 poises) and preferably 0.5 to 25 Pa·s, measured using a Contraves TV or Rheomat 80 viscometer, equipped with a spindle rotating at 60 Hz. A person skilled in the art can select a spindle for measuring the viscosity from the spindles MS-r3 and MS-r4, on the basis of his general knowledge, so as to be able to perform the measurement on a pasty compound.

In exemplary embodiments, fatty substances are hydrocarbon-based compounds optionally of polymeric type; they may also be chosen, for example, from silicone compounds; they may also be in the form of a mixture of hydrocarbon-based and/or silicone compounds. In the case of a mixture of different pasty fatty substances, hydrocarbon-based pasty compounds (mainly containing carbon and hydrogen atoms and optionally ester groups) may be used in a greater proportion.

Exemplary pasty compounds that may be used in foundation-type compositions include, but are not limited to, lanolins and lanolin derivatives, for instance acetylated lanolins, oxypropylenated lanolins or isopropyl lanolate, with a viscosity of from 18 to 21 Pa·s and preferably 19 to 20.5 Pa·s, and/or a melting point of from 30 to 55° C., and mixtures thereof. Esters of fatty acids or of fatty alcohols, especially those containing 20 to 65 carbon atoms (melting point from about 20 to 35° C. and/or viscosity at 40° C. ranging from 0.1 to 40 Pa·s) may also be used, for instance triisostearyl citrate or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixinr” from Rheox.

Exemplary pasty compounds may also include silicone pasty fatty substances such as polydimethylsiloxanes (PDMS) of high molecular weight and in particular those with pendant chains of the alkyl or alkoxy type containing from 8 to 24 carbon atoms, and a melting point of 20-55° C., for instance stearyl dimethicones, especially those sold by the company Dow Corning under the trade names DC2503® and DC25514®, and mixtures thereof.

Pasty fatty substances may be present in exemplary compositions contents ranging from 0.01% to 50% by weight, from 0.1% to 45% by weight and/or from 0.2% to 30% by weight relative to a total weight of a composition.

Exemplary fatty phases may also comprise waxes. Exemplary waxes may be solid at room temperature (25° C.), with a reversible solid/liquid change of state, having a melting point of greater than 30° C. that may be up to 200° C., a hardness of greater than 0.5 MPa and having an anisotropic crystalline organization in the solid state. Exemplary waxes may be hydrocarbon-based waxes, fluoro waxes and/or silicone waxes and may be of animal, plant, mineral or synthetic origin. Exemplary waxes may be chosen, for example, from beeswax, carnauba wax, candelilla wax, paraffin waxes, hydrogenated castor oil, silicone waxes or microcrystalline waxes, and mixtures thereof.

Waxes may be present in the form of a wax-in-water emulsion.

Waxes may be present in exemplary compositions in contents ranging, for example, from 0.01% to 50% by weight, from 0.1% to 30% by weight and/or from 0.2% to 20% by weight relative to a total weight of a composition.

Surfactants

Exemplary emulsions may be stabilized with at least one emulsifying surfactant, which is present, for example, in a proportion ranging from 0.1% to 30% by weight and/or from 5% to 15% by weight relative to a total weight of a composition.

Surfactants may be chosen, for example, from anionic and nonionic surfactants. Reference may be made to the “Encyclopedia of Chemical Technology, Kirk-Othmer,” volume 22, pp. 333-432, 3rd edition, 1979, Wiley, for definitions of the properties and functions (emulsifying) of surfactants, in particular pp. 347-377 of this reference, for anionic and nonionic surfactants.

Exemplary surfactants include, but are not limited to:
nonionic surfactants: fatty acids, fatty alcohols, polyethoxylated or polyglycerolated fatty alcohols such as polyethoxylated stearyl or cetylstearyl alcohol, fatty acid esters of sucrose, alkyl glucose esters, in particular polyoxyethylenated C₁-C₆ alkyl glucose fatty esters, and mixtures thereof;
anionic surfactants:
C₁₆-C₃₀ fatty acids neutralized with amines, aqueous ammonia or alkaline salts, salts of polyoxyethylenated fatty acids, especially those derived from amines or alkaline salts, and mixtures thereof;
phosphoric esters and salts thereof such as “DEA oleth-10 phosphate” (Crodafos N 10 N from the company Croda);
sulfosuccinates such as “Disodium PEG-5 citrate lauryl sulfosuccinate” and “Disodium ricinoleamido MEA sulfosuccinate;”
alkyl ether sulfates such as sodium lauryl ether sulfate;
isethionates; and
acylglutamates such as “Disodium hydrogenated tallow glutamate” (Amisoft HS-21 R sold by the company Ajinomoto) and mixtures thereof.

Surfactants that allow an oil-in-water emulsion to be obtained may preferably be used.

Film-Forming Polymer

Exemplary compositions may also comprise at least one film-forming polymer.

In exemplary embodiments, polymers are present in a continuous phase of an emulsion. Polymers may be present in soluble or dispersed form.

As used herein, the term “film-forming polymer” refers to a polymer capable of forming, by itself or in the presence of an auxiliary film-forming agent, a continuous film that adheres to skin.

A film-forming polymer capable of forming a hydrophobic film, i.e., a polymer whose film has a solubility in water at 25° C. of less than 1% by weight, may preferably be used.

Exemplary film-forming polymers include, but are not limited to:
water-soluble film-forming polymers;
aqueous dispersions of water-dispersible film-forming polymer particles, also known as “lattices;” in such a case, a composition of foundation type should comprise an aqueous phase;
liposoluble film-forming polymers; and
lipodispersible film-forming polymers in the form of nonaqueous dispersions of polymer particles, preferably dispersions of polymer particles, where appropriate surface-stabilized with at least one stabilizer, in one or more silicone and/or hydrocarbon-based oils; these nonaqueous dispersions are also known as “NADs.”

Exemplary compositions, including compositions of foundation type, may comprise a mixture of these polymers.

Film-forming polymers may be present in exemplary compositions in a solid content ranging, for example, from 0.01% to 20% by weight and/or from 0.5% to 10% by weight relative to a total weight of a composition.

Among film-forming polymers that may be used in exemplary compositions, mention may be made of synthetic polymers, of free-radical type or of polycondensate type, polymers of natural origin, and mixtures thereof.

As used herein, the term “free-radical film-forming polymer” refers to a polymer obtained by polymerization of unsaturated monomers, especially ethylenically unsaturated monomers, each monomer being capable of homopolymerizing (unlike polycondensates).

Film-forming polymers of free-radical type may especially include vinyl polymers or copolymers, especially acrylic polymers.

Exemplary film-forming polycondensates include, but are not limited to, polyurethanes, polyesters, polyesteramides, polyamides, epoxyester resins and polyureas.

Polyurethanes may be chosen, for example, from anionic, cationic, nonionic and amphoteric polyurethanes, polyurethane-acrylics, polyurethanes-polyvinylpyrrolidones, polyester-polyurethanes, polyether-polyurethanes, polyureas and polyurea-polyurethanes, and mixtures thereof.

Polymers of natural origin, optionally modified, may be chosen, for example, from shellac resin, sandarac gum, dammar resins, elemi gums, copal resins and cellulose-based polymers, and mixtures thereof.

Film-forming polymers may be present in the form of particles in aqueous dispersion, which are generally known as latices or pseudolatices.

Aqueous dispersions of film-forming polymers that may be used include, but are not limited to, acrylic dispersions sold under the names NEOCRYL XK-90, NEOCRYL A-1070, NEOCRYL A-1090, NEOCRYL BT-62, NEOCRYL A-1079, NEOCRYL A-523 by the company Avecia-Neoresins, DOW LATEX 432 by the company Dow Chemical, DAITOSOL 5000 AD by the company Daito Kasey Kogyo; or alternatively aqueous polyurethane dispersions sold under the names NEOREZ R-981, NEOREZ R-974 by the company Avecia-Neoresins, AVALURE UR-405, AVALURE UR-410, AVALURE UR-425, AVALURE UR-450, SANCURE 875, SANCURE 861, SANCURE 878, SANCURE 2060 by the company Goodrich, IMPRARIL 85 by the company Bayer and AQUAMERE H-1511 by the company Hydromer.

Aqueous dispersions of film-forming polymers that may also be used include, but are not limited to, polymer dispersions resulting from the free-radical polymerization of one or more free-radical monomers within and/or partially at the surface of preexisting particles of at least one polymer chosen from the group consisting of polyurethanes, polyureas, polyesters, polyesteramides and/or alkyds. These polymers are generally known as hybrid polymers.

Film-forming polymers may also be water-soluble polymers and thus may be present in an aqueous phase of a composition in dissolved form. Examples of water-soluble film-forming polymers that may be mentioned include, but are not limited to:
proteins, for instance proteins of plant origin such as wheat or soybean proteins; proteins of animal origin such as keratins, for example keratin hydrolyzates and sulfonic keratins;
anionic, cationic, amphoteric or nonionic chitin or chitosan polymers;
cellulose polymers such as hydroxyethylcellulose, hydroxypropylcellulose, methylcellulose, ethylhydroxyethylcellulose, carboxymethylcellulose and quaternized cellulose derivatives;
acrylic polymers or copolymers, such as polyacrylates or polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of vinylcaprolactam; polyvinyl alcohol;
polymers of natural origin, optionally modified, such as:
gum arabic, guar gum, xanthan derivatives and karaya gum;
alginates and carrageenans;
glycosaminoglycans, and hyaluronic acid and its derivatives;
shellac resin, sandarac gum, dammar resins, elemi gums and copal resins;
deoxyribonucleic acid;
mucopolysaccharides such as hyaluronic acid, chondroitin sulfates;
and mixtures thereof.

In various exemplary embodiments, film-forming polymers may be present in a liquid fatty phase as described above.

Film-forming polymers may also be present in the form of surface-stabilized particles.

Dispersions of surface-stabilized polymer particles may be manufactured as described in EP 749 747.

Polymer particles may be surface-stabilized by means of a stabilizer, which may be a block polymer, a grafted polymer and/or a random polymer, alone or as a mixture.

Dispersions of film-forming polymers in a liquid fatty phase, in the presence of stabilizers, are described, for example, in documents EP 749 746, EP 923 928 and EP 930 060, the disclosures of which are incorporated by reference into the present patent application.

In exemplary embodiments, film-forming polymers may be dissolved in a liquid fatty phase, in which case the film-forming polymer are said to be liposoluble polymers.

Examples of liposoluble polymers that may be mentioned include, but are not limited to, copolymers of a vinyl ester (the vinyl group being directly attached to the oxygen atom of the ester group and the vinyl ester having a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (the alkyl group of which contains from 2 to 18 carbon atoms) or an allylic or methallylic ester (containing a saturated, linear or branched hydrocarbon-based radical of 1 to 19 carbon atoms, linked to the carbonyl of the ester group).

Copolymers may be crosslinked using crosslinking agents, which may be either of the vinyl type or of the allylic or methallylic type, such as tetraallyloxyethane, divinylbenzene, divinyl octanedioate, divinyl dodecanedioate, and divinyl octadecanedioate.

Exemplary copolymers that may be mentioned include, but are not limited to: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate-/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene, and allyl propionate/allyl stearate, crosslinked with 0.2% divinylbenzene.

Liposoluble film-forming polymers that may also be mentioned include, but are not limited to, liposoluble homopolymers, and in particular those resulting from the homopolymerization of vinyl esters containing from 9 to 22 carbon atoms or of alkyl acrylates or methacrylates, the alkyl radicals containing from 10 to 20 carbon atoms.

Such liposoluble homopolymers may be chosen, for example, from polyvinyl stearate, polyvinyl stearate crosslinked with divinylbenzene, with diallyl ether or with diallyl phthalate, polystearyl (meth)acrylate, polyvinyl laurate and polylauryl (meth)acrylate, these poly(meth)acrylates possibly being crosslinked with ethylene glycol dimethacrylate or tetraethylene glycol dimethacrylate.

Such liposoluble copolymers and homopolymers above described, for example, in FR 2 232 303; such liposoluble copolymers and homopolymers may have a weight-average molecular weight ranging, for example, from 2,000 to 500,000 and/or from 4,000 to 200,000.

Exemplary liposoluble film-forming polymers include, but are not limited to, polyalkylenes and especially copolymers of C₂-C₂₀ alkenes, for instance polybutene, alkylcelluloses with a saturated or unsaturated, linear or branched C₁ to C₈ alkyl radical, for instance ethylcellulose and propylcellulose, vinylpyrrolidone (VP) copolymers and especially copolymers of vinylpyrrolidone and of a C₂ to C₄₀ and better still C₃ to C₂₀ alkene. Exemplary VP copolymers include, but are not limited to, VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate copolymer.

Exemplary compositions may also comprise an auxiliary film-forming agent that promotes formation of a film with a film-forming polymer. Such film-forming agents may be chosen from any compound known to the those skilled in the art as being capable of satisfying the desired function, and may be chosen especially from plasticizers and coalescers.

Other Dyestuffs

As used herein, “dyestuffs” refers to dyestuffs that are different from dyes with a liquid-crystal colored phase and may include, for example, any compound chosen from liposoluble and water-soluble monochromatic dyes, nacres and reflective particles.

Amounts of additional dyestuff may be selected so as not to adversely affect an optical effect afforded by a dye with a liquid-crystal colored phase.

Liposoluble and water-soluble monochromatic dyes:

Exemplary compositions may comprise at least one monochromatic dye, such as a natural organic dye such as cochineal carmine, and/or a synthetic dye such as halo acid dyes, azo dyes or anthraquinone dyes. Mention may also be made, for example, of mineral dyes such as copper sulfate or iron sulfate. Mention may also be made, for example, of Sudan brown, Sudan red and annatto, and also beetroot juice, carotene and methylene blue.

Additional dyestuffs may be present in exemplary compositions alone or as a mixture, in proportions ranging, for example, from 0.001% to 15% by weight, from 0.01% to 5% by weight and/or from 0.1% to 2% by weight, relative to the total weight of said composition.

Pigments:

“Pigments,” as used herein, refers to white or colored, mineral or organic particles intended to color and/or opacify a composition.

Pigments may be present in exemplary compositions in a proportions, for example, of from 0.01% to 25% by weight and/or from 3% to 10% by weight of a composition.

Pigments may be white or colored, mineral or organic, and of standard or nanometric size. They may be in the form of powder or of pigmentary paste. Mention may be made, for example, of titanium oxide, zirconium oxide or cerium oxide, and also zinc oxide, iron oxide, or chromium oxide, ferric blue, chromium hydrate, carbon black, ultramarines (aluminosilicate polysulfides), manganese pyrophosphate and certain metal powders such as silver or aluminum powder. Mention may also be made, for example, of D&C pigments and lakes commonly used to give the lips and the skin a makeup effect, which include calcium, barium, aluminum, strontium or zirconium salts.

Nacres:

“Nacres,” as used herein, refers to iridescent particles that reflect light, which are produced especially by certain molluscs in their shell, or alternatively which are synthesized.

Nacres may be present in exemplary first and/or second composition(s) especially in proportions of from 0.01% to 20% by weight and/or of about from 3% to 10% by weight relative to a total weight of a composition. Among nacres that may be employed, mention may be made, for example, of natural nacre, mica coated with titanium oxide, with iron oxide, with natural pigment or with bismuth oxychloride, and also colored titanium mica.

Additives:

Exemplary compositions may also contain one or more cosmetic, dermatological, hygiene or pharmaceutical active agents.

Cosmetic, dermatological, hygiene or pharmaceutical active agents may include, but are not limited to, moisturizers (polyols, for instance glycerol), vitamins (C, A, E, F, B or PP), essential fatty acids, essential oils, ceramides, sphingolipids, liposoluble sunscreens or sunscreens in the form of nanoparticles, and specific active agents for treating the skin (protective agents, antibacterial agents, anti-wrinkle agents, etc.).

Active agents may be used in usual amounts known to those skilled in the art such as, for example, concentrations of from 0 to 20% and/or from 0.001% to 15% of a total weight of a composition.

Exemplary compositions may furthermore comprise, depending on the type of application envisioned, constituents conventionally used in fields under consideration, which are present in an amount that is suitable for the desired galenical form.

Exemplary compositions may be colored makeup compositions, for example complexion products (foundations), makeup rouges, eyeshadows, lip products, concealer products, blushers, mascaras, eyeliners, eyebrow makeup products, lip or eye pencils, nail products, body makeup products or hair makeup products (hair mascara or hair lacquer).

Exemplary compositions may be nail varnishes.

Exemplary compositions may be used in unmodified form for application, or may be combined with another mode of makeup already applied. When exemplary compositions are applied as surface products, they may features a “top coat.”

Exemplary compositions or kits may be applied to makeup accessories (supports), such as false nails, false eyelashes, wigs or hairpieces, or alternatively to dots or patches adhering to the skin or the lips (of the beauty spot type).

In various exemplary embodiments of kits according to the present invention, a second composition combined with an exemplary composition according to the invention, especially a base composition and/or a surface composition, may be in a solid form, for example pulverulent, compacted or cast form, or in the form of a stick or in the form of a fluid, for example a pasty or liquid fluid. Exemplary compositions may also be in the form of a soft paste, an ointment or a solid or fluid pomade of cream type. For example, exemplary compositions may be oil-in-water or water-in-oil emulsions, solid or soft gels, especially anhydrous gels, which may be in a two-phase form.

Exemplary compositions may contain at least one dyestuff especially as defined above, formulated in a physiologically acceptable medium.

The term “physiologically acceptable medium,” as used herein, refers to a nontoxic medium that may be applied to human skin, mucous membranes or integuments. A physiologically acceptable medium is generally suited to the nature of the support onto which the composition is to be applied, for instance the skin or the integuments, and also to the form in which the composition is intended to be packaged, e.g., a fluid at room temperature and at atmospheric pressure.

Exemplary surface compositions may be formulated in a fluid or solid form of loose, compact or cast powder type.

Exemplary compositions may be in an anhydrous form or in the form of a gel, a direct, inverse or multiple emulsion combining at least one aqueous phase and at least one fatty phase.

In exemplary embodiments of kits according to the present invention, two corresponding compositions may be in identical or different forms, and especially as described herein.

This invention is illustrated by the following examples, which are merely for the purpose of illustration.

### EXAMPLES

### Example 1

Various emulsions are prepared with a dye of liquid-crystal type containing a chiral nematic phase. The dye used is sold under the trade name BN 600 by the company Hallcrest.

The compositions of the various emulsions prepared are indicated in the table below. These emulsions are prepared according to the following procedure.

The dye with a liquid-crystal colored phase is dissolved in isododecane in the proportions indicated. Polyethylene glycol monoisostearate is added thereto as surfactant when the dissolution of the dye is complete. The mixture is stirred until a homogeneous solution is obtained.

In parallel, an aqueous phase is prepared by dissolving 10% polyvinyl alcohol in water with heating. The mixture is allowed to cool to room temperature. 10 g of corresponding emulsion are prepared in a test tube by first introducing the isododecane-based solution into the tube. The mixture is shaken by hand using a flat spherical stainless-steel shaker with small holes, and the aqueous phase is introduced therein dropwise with stirring and at room temperature. The emulsions thus obtained rest for at least two days without demixing.

TABLE

| Test No. | 1 | 2 | 3 | 4 | 5 | 6 |
|---|---|---|---|---|---|---|
| Isododecane | 15 | 15 | 15 | 15 | 15 | 20.5 |
| Dye BN 600 | 30 | 20 | 15 | 12 | 10 | 9.5 |
| Surfactant = PEG-8 | 5 | 5 | 5 | 5 | 5 | 5 |
| monoisostearate | | | | | | |
| 10% solution of | 53 | 60 | 65 | 68 | 70 | 65 |
| polyvinyl alcohol | | | | | | |
| in H₂O | | | | | | |
| Amount | 40.3 | 31 | 26.5 | 23.8 | 22 | 21 |
| remaining after | | | | | | |
| evaporation of | | | | | | |
| H₂O and | | | | | | |
| isododecane | | | | | | |
| Weight | 74.4% | 64.5% | 56.6% | 50.4% | 45.5% | 45.2% |
| concentration of | | | | | | |
| LC in the deposit | | | | | | |
| after evaporation | | | | | | |
| Observation of | Colors and | Colors and | No visual | No visual | No visual | No visual |
| the deposit | iridescence | iridescence | effects | effects | effects | effects |
| visually with | | | | | | |
| white light | | | | | | |
| Observation | Birefringence | Birefringence | Birefringence | Birefringence | No more | No more |
| using a | | | | limit | polarizing | polarizing |
| polarized-light | | | | | effects | effects |
| microscope* | | | | | | |

*Leica DMLB microscope

Only tests 1, 2 and 4 lead to deposits showing optical effects. These deposits are moreover free of tacky feel and the corresponding cosmetic properties are satisfactory.

### Example 2

An emulsion is prepared by using as a dye with a liquid-crystal nematic phase BN 825 sold by the company Hallcrest.

This dye is added in a proportion of 30 g to 15 g of isododecane according to the protocol described above; this oily phase also comprises the above-mentioned surfactant in a proportion of 5 g.

This aqueous phase is mixed with 53 g of a 10% solution of polyvinyl alcohol in H₂O, according to the protocol described previously.

The deposit obtained after application and drying of the emulsion comprises 74.4% by weight of liquid crystals.

This deposit shows iridescent colors and is characterized, by polarized-light microscopy, by birefringence.

The deposit is also free of any coloring effect and shows satisfactory cosmetic properties.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A emulsion-type cosmetic composition for caring for and/or making up the skin, mucous membranes and/or the integuments, comprising:
at least one liquid continuous phase; and
at least one liquid dispersed phase;
wherein:
the composition comprises at least one volatile solvent;
the dispersed liquid phase comprises at least one dye with a liquid-crystal colored phase; and
the at least one dye is present in a concentration such that, after the composition is dried on a support, a dried concentration of the dye is greater than or equal to about 50% by weight relative to a total weight of the dried composition.

2. The composition of claim 1, wherein the at least one dye has a chiral nematic phase.

3. The composition of claim 1, wherein the at least one dye is heat-sensitive.

4. The composition of claim 1, wherein the at least one dye is present in the at least one dispersed liquid phase in dissolved form.

5. The composition of claim 1, wherein the at least one dye is dissolved in the at least one volatile solvent.

6. The composition of claim 1, wherein the at least one liquid continuous phase comprises the at least one volatile solvent.

7. The composition of claim 1, wherein the at least one liquid continuous phase comprises at least one film-forming compound.

8. The composition of claim 1, wherein the composition is in the form of an oil-in-water emulsion.

9. The composition of claim 1, wherein the composition is in the form of an oil-in-oil emulsion.

10. The composition of claim 9, wherein a first phase of the oil-in-oil emulsuion comprises at least one hydrocarbon-based oil and a second phase of the oil-in-oil emulsion comprises at least one silicone oil.

11. The composition of claim 1, wherein the composition in the form of a water-in-oil emulsion.

12. The composition of claim 11, wherein an aqueous phase of the water-in-oil emulsion is an aqueous-alcoholic phase.

13. The composition of claim 1, wherein the at least one dye is at least one of a liposoluble dye with a chiral nematic liquid-crystal colored phase or an organosoluble dye with a chiral nematic liquid-crystal colored phase.

14. The composition of claim 1, wherein the at least one dye comprises synthetic liquid crystals.

15. The composition of claim 1, wherein the concentration of the at least one dye is such that, after the composition is dried on the support, the dried concentration of the dye is greater than or equal to 60% by weight relative to the total weight of the dried composition.

16. The composition of claim 1, wherein the concentration of the at least one dye is such that, after the composition is dried on the support, the dried concentration of the dye is from about 60% to about 80% by weight relative to the total weight of the dried composition.

17. The composition of claim 1, wherein the concentration of the at least one dye is such that, after the composition is dried on the support, the dried concentration of the dye is from about 64% to about 75% by weight relative to the total weight of the dried composition.

18. The composition of claim 1, further comprising at least one additional dyestuff.

19. The composition of claim 18, wherein at least one additional dyestuff is present in the at least one liquid continuous phase.

20. The composition of claim 18, wherein the at least one dye is present in the composition in a concentration of at least about 60% by weight relative to a total weight of the at least one dye and the at least one additional dyestuff.

21. The composition of claim 18, wherein the at least one additional dyestuff is present in the composition in a concentration of about 10% by weight or less relative to a total weight of the at least one dye and the at least one additional dyestuff.

22. The composition of claim 1, wherein the composition comprises at least one liquid fatty phase.

23. The composition of claim 22, wherein the liquid fatty phase comprises at least one volatile or nonvolatile oil.

24. The composition of claim 23, wherein the at least one volatile or nonvolatile oil comprises at least one hydrocarbon-based oil.

25. The composition of claim 23, wherein the at least one volatile or nonvolatile oil comprises at least one member selected from the group consisting of animal oils, plant oils, synthetic esters, synthetic ethers, fatty acids, fatty alcohols, linear hydrocarbons of mineral origin, branched hydrocarbons of mineral origin, linear hydrocarbons of synthetic origin, branched hydrocarbons of synthetic origin, and mixtures thereof.

26. The composition of claim 22, wherein the liquid fatty phase is present in the composition at a concentration of from about 0.01% to about 90% by weight relative to a total weight of the composition.

27. The composition of claim 22, wherein the liquid fatty phase is present in the composition at a concentration of from about 0.1% to about 85% by weight relative to a total weight of the composition.

28. The composition of claim 1, wherein the composition comprises at least one aqueous phase.

29. The composition of claim 1, wherein the composition comprises at least one surfactant.

30. The composition of claim 30, wherein the at least one surfactant is present in the composition at a concentration of from about 0.1% to about 30% by weight relative to a total weight of the composition

31. The composition of claim 1, wherein the composition is a nail varnish.

32. A process for making up the skin, the lips and/or the integuments, comprising applying the composition of claim 1 to the skin, the lips and/or the integuments.

33. A cosmetic process for making up the skin, mucous membranes and/or the integuments, comprising:
applying a first coat of a first cosmetic composition to a surface to be made up; and
applying a second coat of a second cosmetic composition over the first coat;
wherein:
the first cosmetic composition comprises at least one dyestuff in a physiologically acceptable medium, the first cosmetic composition being free of dyes with liquid-crystal colored phases; and
the second cosmetic composition is an emulsion-type composition comprising at least one liquid continuous phase and at least one liquid dispersed phase, the second cosmetic composition comprising at least one volatile solvent and the liquid dispersed phase comprising an effective amount of at least one dye with a liquid-crystal colored phase.

34. The process of claim 33, wherein the second cosmetic composition includes the at least one dye in a concentration such that, after the second cosmetic composition is dried on a support, a dried concentration of the dye is greater than or equal to about 50% by weight relative to a total weight of the dried second cosmetic composition.

35. A makeup kit, comprising:
a first cosmetic composition comprising at least one dyestuff in a cosmetically acceptable medium, the first composition being free of dyes with liquid-crystal colored phases; and
a second emulsion-type cosmetic composition comprising at least one liquid continuous phase and at least one liquid dispersed phase, the second cosmetic composition comprising at least one volatile solvent and the liquid dispersed phase comprising an effective amount of at least one dye with a liquid-crystal colored phase;
wherein the first and second compositions are packaged in separate containers.

36. The kit of claim 35, wherein the second cosmetic composition includes the at least one dye in a concentration such that, after the second cosmetic composition is dried on a support, a dried concentration of the dye is greater than or equal to about 50% by weight relative to a total weight of the dried second cosmetic composition.

37. A made-up support, comprising a support having the composition of claim 1 applied thereto, wherein the support is selected from the group consisting of false nails, false eyelashes, wigs, hairpieces, dots that adhere to the skin or the lips, and patches that adhere to the skin or the lips.
